(19) United States
(12) Patent Application Publication
(10) Pub. No.: US 2026/0204358 A1
(43) Pub. Date: Jul. 16, 2026

(54) METHOD AND IDENTIFICATION SYSTEM FOR NONTARGET IDENTIFICATION OF PER- AND POLYFLUOROALKYL SUBSTANCES

(71) Applicant: NANJING UNIVERSITY, NANJING (CN)

(72) Inventors: NANYANG YU, NANJING (CN); LAIHUI LI, NANJING (CN); KAN XU, NANJING (CN); XUEBING WANG, NANJING (CN); YIYAN DENG, NANJING (CN); SI WEI, NANJING (CN)

(73) Assignee: NANJING UNIVERSITY, NANJING (CN)

(21) Appl. No.: 18/819,027

(22) Filed: Aug. 29, 2024

(30) Foreign Application Priority Data
Aug. 30, 2023 (CN) 202311102756.2

(51) Int. Cl. G16C 20/20; G16C 20/80

(52) U.S. Cl. CPC G16C 20/20; G16C 20/80

(57) ABSTRACT

This disclosure provides a nontarget identification method and an identification system for per- and polyfluoroalkyl substances homologues. The method includes: performing deconvolution on mass spectrometry sample data (in SWATH acquisition mode) by a sample correlation-based deconvolution algorithm; extracting per- and polyfluoroalkyl substances in sample peaks according to homologue-specific rules; and identifying PFASs by MetFrag after ion annotation of these sample peaks. The method of the disclosure improves the identification effect, demonstrating that the SWATH-F technique proposed in the disclosure is an effective tool for the detection of PFASs in environmental and human samples.

### CROSS-REFERENCE OF RELATED APPLICATION

This application claims priority of Chinese Patent Application No. 202311102756.2, filed on Aug. 30, 2023, the contents of which are hereby incorporated by reference.

### TECHNICAL FIELD

This disclosure relates to the field of the homologues identification, in particular to a method and an identification system for nontarget identification of per- and polyfluoroalkyl substances.

### BACKGROUND

In environmental mass spectrometry analysis, the predominant method for high-throughput acquisition of secondary mass spectra (MS2) is currently the Information Dependent Acquisition (IDA) mode. The nontarget analysis of the Information Dependent Acquisition (IDA) mode using high-resolution mass spectrometry in IDA mode is crucial for analyzing per- and polyfluoroalkyl substances (PFASs), serving as an effective tool in the discovery of novel PFASs. However, the spectral information yielded by IDA is quite limited, capturing only high-concentration PFASs. Given that PFASs are trace-level pollutants found in the environment and biological systems, and exhibit a broad diversity, the challenge in identifying the profile of all PFASs is significant.

Some reports have confirmed the presence of numerous novel PFASs with unknown structures in the environment. The sequential window acquisition of all theoretical fragment-ion spectra (SWATH mode), though extensively employed in proteomics for capturing MS2 spectral data of all precursor ions simultaneously, struggles with the complexity of mass spectrometry data and background noise, making direct identification of PFASs challenging. Additionally, the lack of pre-existing knowledge on novel per- and polyfluoroalkyl substances hinders the identification and structural determination of PFASs. As a result, it is difficult for nontargeted screening methods to be applied to the identification of PFASs substances.

In view of this, the disclosure is provided.

### SUMMARY

In view of this, this disclosure provides a method and an identification system for nontarget identification of homologues. By comparing the screening results of nontarget homologues by the SWATH-F method with a classical IDA method in collection mode, the results show that the identification effect of SWATH-F is better. The comparison highlights SWATH-F's superior performance, with an identification increase of 276% (from 17 classes of PFASs with IDA to 64 classes with SWATH-F). Furthermore, SWATH-F's screening and identification processes have been automated, making it a convenient and effective tool for researchers studying PFASs in environmental and biological samples.

Specifically, the disclosure is realized through the following technical solutions.

In the first aspect, a method for nontarget identification of perfluoroalkyl (PFAS) homologues is provided, which includes the following steps:
performing deconvolution on sample data from mass spectrometry by a sample correlation-based deconvolution algorithm, extracting sample peaks according to homologue rules, identifying PFASs by MetFrag after ion annotation being performed on the sample peaks.

In the second aspect, an identification system for nontarget identification of PFAS homologues, which includes:
an extraction module: configured for performing deconvolution on sample data from mass spectrometry by the sample correlation-based deconvolution algorithm and extracting the sample peaks according to the homologue rule:
an identification module: configured for identifying the perfluoroalkyl substances PFASs by MetFrag after ion annotation being performed on the sample peaks.

In the third aspect, the disclosure provides a computer-readable storage medium on which a computer program is stored, where when the program is executed, steps of the method for nontarget identification of homologues as described in the second aspect are implemented.

In the fourth aspect, the disclosure provides a computer device, including a memory, a processor, and a computer program stored on the memory and executable on the processor, where when the processor executes the program, steps of the method for nontarget identification of homologues as described in the second aspect are implemented.

This method and system for nontarget identification of homologues, a novel nontarget screening strategy-SWATHF based on a SWATH-MS deconvolution method is realized, which is used for solving the problem that the accuracy of accurately annotating PFAS homologues in multi-sample research is not high. It is realized that PFAS homologues can be screened from multiple samples (not less than five) based on homologue rules, and clean MS2 spectrograms with characteristic structural fragments can be provided, which can accurately identify and annotate PFASs homologues in human serum studies with high confidence.

### BRIEF DESCRIPTION OF THE DRAWINGS

Various other advantages and benefits will become clear to those skilled in the field by reading the following detailed description of the preferred embodiments. The drawings are only for the purpose of illustrating preferred embodiments and are not considered as limiting the scope of the disclosure. Moreover, the same parts are represented by the same reference numbers throughout the drawings. In the attached drawings:

FIG. 1 is a specific flowchart diagram of a method for nontarget identification of homologues according to an embodiment of this disclosure;

FIG. 2a is a result diagram showing the detection rate (DR) and MS2 generation rate (MS2) for samples with gradient additions of PFAS standards from embodiment 1 according to this disclosure:

FIG. 2b is a result diagram showing the relative standard deviation (RSD) of PFAS internal standard peak areas from embodiment 1 according to this disclosure, where dark columns indicate results from the IDA method and light columns represent those from the SWATH-F method;

FIG. 3 shows categories of the different homologue types identified in embodiment 1 according to this disclosure.

FIG. 4 is a flowchart diagram of the computer device according to an embodiment of the disclosure.

### DETAILED DESCRIPTION OF PREFERRED EMBODIMENTS

Illustrative embodiments will be illustrated in detail here, examples of which are shown in the attached figure. When the following description refers to the drawings, unless otherwise indicated, the same numbers in different drawings indicate the same or similar elements. The embodiments described in the following exemplary embodiments do not represent all embodiments consistent with the disclosure. Rather, they are merely examples of devices and methods consistent with some aspects of the disclosure as detailed in the appended claims.

The terminology employed herein aims to describe particular implementations without imposing limitations on the scope of the disclosure. The singular forms “a”, “the” and “the” used in this disclosure and the appended claims are also intended to include the plural forms, unless the context clearly indicates other meaning. It should also be understood that the term “and/or” as used herein refers to and includes any or all possible combinations of one or more associated listed items.

It should be understood that although the terms first, second, third, etc. may be used to describe various information in this disclosure, such information should not be limited to these terms. These terms are only used to distinguish the same type of information from each other. For example, without departing from the scope of this disclosure, the first information may also be called the second information, and similarly, the second information may also be called the first information. Depending on the context, the word “if” as used herein can be interpreted as “in . . . when” or “when . . . ” or “in response to a determination”.

Referring to FIG. 1, a method for nontarget identification of homologues is provided, which includes:
deconvolution is performed on sample data from mass spectrometry by a sample correlation-based deconvolution algorithm, sample peaks are extracted according to homologue rule, perfluoroalkyl substances PFASs are identified by MetFrag after ion annotation is performed on the sample peaks.

### Embodiment 1

The specific operation steps are as follows, as shown in FIG. 1:
first, sample preprocessing: the process of sample storage and preparation is based on the previously published method, which is an improved QuEChERS technology suitable for blood sample preparation.
second, instrumental analysis: IDA and SWATH analysis detection are successively performed on the preprocessed samples by UPLC-QTOF-MS liquid chromatography-mass spectrometry system (SCIEX, USA). The setting parameters of the instrument are:
chromatograph: Ultimate 3000 ultra-high efficiency liquid chromatograph;
chromatograph column: C18 column (2.1 mm×50 mm, 2.5 μm);
column temperature: 40° C.;
flow rate: 0.4 mL/min;
mobile phase in negative ion mode: 2 mM ammonium acetate aqueous solution (Phase A) and methanol (Phase B), as shown in Table 1 below.

TABLE 1
parameter setting results

| | | | flow | inject |
|---|---|---|---|---|
| time (min) | phase A (%) | phase B (%) | rate(mL/min) | volume (μL) |
| 0 | 95 | 5 | 0.4 | 10 |
| 1 | 95 | 5 | 0.4 | 10 |
| 10 | 75 | 25 | 0.4 | 10 |
| 19 | 50 | 50 | 0.4 | 10 |
| 29 | 25 | 75 | 0.4 | 10 |
| 39 | 0 | 100 | 0.4 | 10 |
| 48 | 0 | 100 | 0.4 | 10 |
| 48.1 | 95 | 5 | 0.4 | 10 |
| 55 | 95 | 5 | 0.4 | 10 |

mass spectrometer: QTOF-MS quadrupole-time of light-mass spectrometry.
SWATH mode parameters are as follows:
singlel scan time: 1.020 seconds,
mass range (Da): 99.5 to 1250 Da (M81), 30 to 1250 Da (MS2),
collision energy: −40 electron volts and +20 electron volts (eV),
ion source temperature: 550° C.

The SWATH mode variable window parameters are set as shown in Table 2:

TABLE 2
window parameter setting results

| window | | | |
|---|---|---|---|
| Number | MS Type | Min m/z | Max m/z |
| 0 | SCAN | 99.5 | 1250 |
| 1 | SWATH | 99.5 | 210.3 |
| 2 | SWATH | 209.3 | 242.5 |
| 3 | SWATH | 241.5 | 258.6 |
| 4 | SWATH | 257.6 | 273.0 |
| 5 | SWATH | 272.0 | 286.2 |
| 6 | SWATH | 285.2 | 299.4 |
| 7 | SWATH | 298.4 | 312.7 |
| 8 | SWATH | 311.7 | 329.9 |
| 9 | SWATH | 328.9 | 355.2 |
| 10 | SWATH | 354.2 | 390.3 |
| 11 | SWATH | 389.3 | 434.0 |
| 12 | SWATH | 433.0 | 482.9 |
| 13 | SWATH | 481.9 | 531.2 |
| 14 | SWATH | 530.2 | 568.0 |
| 15 | SWATH | 567.0 | 608.8 |
| 16 | SWATH | 607.8 | 684.1 |
| 17 | SWATH | 683.1 | 768.7 |
| 18 | SWATH | 767.7 | 841.1 |
| 19 | SWATH | 840.1 | 935.4 |
| 20 | SWATH | 934.4 | 1250.0 |

third, screening PFAS homologues by the SWATH-F workflow:
step 1: deconvolution in SWATH-MS: deconvolution is performed on the sample data obtained by SWATH mode by the deconvolution algorithm based on the correlation between samples. For accurate analysis, mass spectrometry peaks across all samples are aligned with a tolerance of 0.5 minutes for retention time (RT) and 0.025 Da for the first mass spectrometry level (M81). The specific parameters are:

TABLE 3
the parameters settings

| parameters | value |
|---|---|
| MS2 tolerance | 0.01 |
| min MS2 peak intensity | 300 |
| min number of detected samples | 3 |
| exclude highest correlated peaks | 0.9 |
| min correlation coefficient (MS2) | 0.7 |
| margin 1(target precursor) | 0.2 |
| margin 2(coeluted precursor) | 0.1 |
| min detected rate | 0.1 |
| min MS2 relative intensity | 0.02 |

step 2: PFAS homologue peak extraction: the MS-DIAL software (version 4.12 or above) is utilized to extract peaks from .wiff2 files produced by the X500R instrument, the peak extraction parameters are set as followings:
signal-to-noise ratio (S/N) is set to 3,
M81 mass accuracy threshold is set to less than 0.01 Da,
ion addion forms includes [M-H]-, [M-H20-H], [M+Na-2H], [M+Cl]-, and [2M-H]-;
retention time tolerances of peak alignment are set to less than 0.5 minutes and the M81 tolerance of peak alignment is less than 0.025 Da. The peak area of serum samples is subtracted from the average peak area of blank program samples for semi-quantitative analysis.
self-developed Python script SWATH-F extracts suspicious PFASs homologues; the calculation rules are as follows: firstly, peaks that exhibit a mass difference equivalent to (CF2)n, (CF2O)n, or (CH2CF2)n (with a mass accuracy within 5 ppm) are preserved as homologue candidates; secondly, each homologue series should observe a progressive increase in m/z and retention time (RT); thirdly, peaks that significantly deviate in mass, indicated by decimal values either greater than 0.85 or less than 0.15, are kept for further examination based on the established mass deficit guidelines of PFAS structural units;
step 3: ion annotation for PFAS: for the peaks extracted in step 2, ion annotation is performed using the SWATH-F workflow; this process involves matching against a database that includes 3496 PFASs and 389 MS2 spectra, yielding 91 characteristic ions, 7 auxiliary identification ions, and 9 types of neutral losses; unknown structures are then predicted with the aid of CFM-ID (v 3.0) and SIRIUS (v 4.0.1), the parameters of matching characteristic ions are set as follows:
mass error: 20 ppm;
step 4: structural identification of PFAS: Utilizing MetFrag (Web), PFASs are identified by leveraging both MS1 and MS2 data. Annotated MS2 spectra and retention times are critically analyzed to filter out false-positive results caused by in-source fragmentation, the specific parameters are set as follows:
mass error: 5 ppm;
retention time tolerance: 0.1 min.

Among the above methods, SWATH+ deconvolution method based on the correlation between samples, and homologue screening (peak extraction), and ion annotation method improve the identification effect. The problem that the deconvolution algorithm in the prior art can not well distinguish the target ion from the noise is abandoned. This specific method of identifying homologues is obtained through a lot of creative efforts, and the specific operation steps and the operation sequence between steps are specific. Only by operating according to the scheme of the disclosure can the ideal identification effect of PFASs be finally obtained. In order to verify the advantages of the method of the disclosure, SWATH-F and IDA acquisition modes are adopted for comparison. The specific experimental process and the experimental results after the comparison of the two acquisition modes are shown in the following experiment 1.

### Experiment 1.

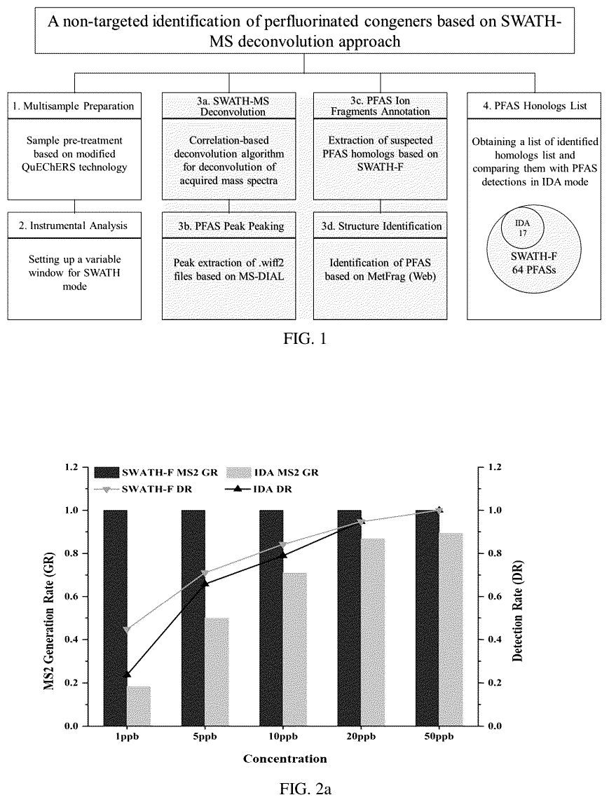

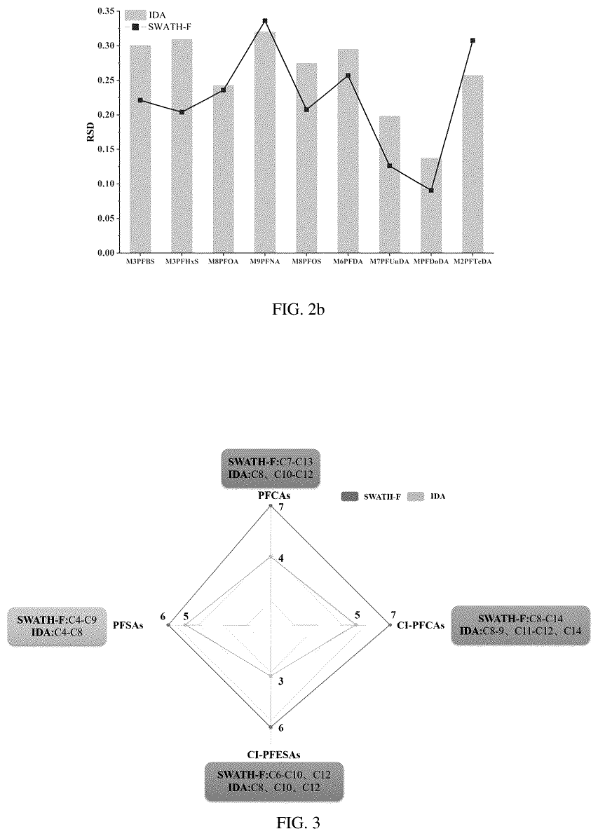

First, gradient spiked sample testing: fetal bovine serum (FBS) is spiked with 38 PFAS standards with five concentrations (1 ppb, 5 ppb, 10 ppb, 20 ppb, 50 ppb), along with nine PFAS internal standards at 20 ppb (see Table 4). Notable differences in MS2 generation and detection rates are observed between IDA and SWATH acquisition modes. At 50 ppb, both modes achieved a 100% detection rate. However, at lower concentrations, IDA mode's ability to capture standard information is impeded by the complex matrix, whereas SWATH mode showed superior performance (see FIG. 2a). Moreover, SWATH mode demonstrated a 100% MS2 generation rate across various FBS standard mixtures at matching mass and retention times. However, the MS2 production rate in IDA mode has a high limit on the concentration of the mixture (see FIG. 2a). By analyzing the relative standard deviation (RSD) of internal standard in parallel determination, the accuracy of the two acquisition modes in multi-sample experiments is evaluated. Nine stable isotope labeled standards of PFAS are added to each sample to correct matrix effect and instrument change. The results show that the RSD values of most PFASs standard abundances in SWATH acquisition mode are less than those in IDA acquisition mode (see FIG. 2b), indicating that SWATH acquisition mode has good data reproducibility.

Second, real population serum sample testing: serum samples from 11 residents near the Fluorochemical Park is screened for PFASs. According to the homologous screening rules, fourteen PFAS series are identified by screening for homologous structural units CF2- and CF2O— in sample peaks, including PFCAs, PFSAs, Cl-PFESAs, Cl-PFCAs, H, Cl-PFCAs, PFOTs, PFOHs, PFEAs, Po-PFEAs, Po-PFECAs, H—Po-PFECAs, and three series of molecular formulas determined by the homologous repeating structural unit CF2O.

In this experiment, both the SWATH-F method and a nontarget PFAS homologue screening script based on the IDA mode detect PFCAs, PFSAs, Cl-PFESAs, and Cl-PFCAs in population serum. However, the coverage rate of PFASs detected by SWATH-F method is higher than that by IDA method, and the classification by different homologues is shown in FIG. 3.

Interestingly, while short-chain Cl-PFCAs (C8-C10) are previously detected in human blood and long-chain Cl-PFCAs (C11-C14) in the environment, the SWATH-F method detected both in human serum. Additionally, substances such as H, Cl-PFCAs, and PFOTs, previously unreported in the environment, are identified, suggesting novel and diverse PFASs presence in the serum of residents living near the Fluorochemical Park. The detected PFAS homologues and their confidence levels by SWATH-F are detailed in the following table:

TABLE 4
experiment result.

| | PFAS | Precursor | RT | MS2_match | | Confidence |
|---|---|---|---|---|---|---|
| Class | Homologues | ions m/z | (min) | fragments | Formula | level |
| 1 | PFCAs | 362.9662 | 17.95 | C₂F₅—/C₃F₇—/C₄F₉— | C₇HF₁₃O₂ | 1 |
| | | 412.9676 | 20.30 | C₂F₅—/C₃F₇—/C₄F₉—/C₇F₁₅— | C₈HF₁₅O₂ | 1 |
| | | 462.9632 | 22.20 | C₂F₅—/C₃F₇— | C₉HF₁₇O₂ | 1 |
| | | 512.9601 | 23.81 | C₂F₅—/C₃F₇—/C₄F₉—/C₅F₁₁—/ | C₁₀HF₁₉O₂ | 1 |
| | | | | C₉F₁₉— | | |
| | | 562.9579 | 24.82 | C₄F₉—/C₁₀F₂₁— | C₁₁HF₂₁O₂ | 1 |
| | | 612.9518 | 26.31 | C₂F₅—/C₃F₇—/C₄F₉—/C₅F₁₁—/ | C1₂HF₂₃O₂ | 1 |
| | | | | C₆F₁₃—/C₁₁F₂₃— | | |
| | | 662.9473 | 27.31 | C₂F₅—/C₃F₇—/C₄F₉/—C5F₁₁—/ | C₁₃HF₂₅O₂ | 1 |
| | | | | C₆F₁₃—/C₇F₁₅—/C₈F₁₇— | | |
| 2 | PFSAs | 298.9416 | 11.09 | No matched | C₄HF₉SO₃ | 1 |
| | | 348.9382 | 15.61 | | C₅HF₁₁SO₃ | 1 |
| | | 398.9374 | 18.47 | CF₃—/SO₃—/FSO₃—/C₂F₅— | C₆HF₁₃SO₃ | 1 |
| | | 448.9338 | 20.60 | CF₃—/SO₃—/FSO₃—/C₂F₅— | C₇HF₁₅SO₃ | 1 |
| | | 498.9289 | 21.71 | SO₃—/FSO₃—/C₂F₅—/CF₂SO₃—/ | C₈HF₁₇SO₃ | 1 |
| | | | | C₃F₇—/C₂F₄SO₃—/C₃F₆SO₃—/ | | |
| | | | | C₄F₈SO₃—/C₅F₁₀SO₃— | | |
| | | 548.9238 | 23.45 | CF₃—/FSO₂—/C₄F₈SO₃— | C₉HF₁₉SO₃ | 1 |
| 3 | Cl—PFCAs | 428.9362 | 20.73 | Cl—/CF₃—/C₄F₉O— | C₈HF₁₄ClO₂ | 2 |
| | | 478.9326 | 22.22 | Cl—/C₂F₅—/C₃F₇—/C₄F₉—/ | C₉HF₁₆ClO₂ | 1 |
| | | | | C₅F₁₁—/C₅F₁₀Cl— | | |
| | | 528.9296 | 24.11 | Cl— | C₁₀HF₁₈ClO₂ | 2 |
| | | 578.9249 | 25.44 | Cl—/C₈F₁₆Cl— | C₁₁HF₂₀ClO₂ | 2 |
| | | 628.9208 | 26.54 | Cl—/C₃F₇— | C₁₂HF₂₂ClO₂ | 2 |
| | | 678.9233 | 27.51 | Cl— | C₁₃HF₂₄ClO₂ | 2 |
| | | 728.9123 | 28.28 | Cl— | C₁₄HF₂₆ClO₂ | 2 |
| 4 | Cl—PFESAs | 430.8996 | 19.80 | Cl— | C₆HClF₁₂O₄S | 2 |
| | | 480.8987 | 21.66 | Cl—/FSO₃— | C₇HClF₁₄O₄S | 2 |
| | | 530.8932 | 23.26 | Cl—/FSO₂—/FSO₃— | C₈HClF₁₆O₄S | 1 |
| | | 580.8934 | 24.66 | Cl— | C₉HClF₁₈O₄S | 2 |
| | | 630.8890 | 25.84 | Cl—/FSO₂—/FSO₃—/C₂F₅SO₃—/ | C₁₀HClF₂₀O₄S | 1 |
| | | | | C₈F₁₆ClO—/C₁₀F₂₀ClSO₄— | | |
| | | 730.8824 | 27.76 | C₁₂F24ClSO₄— | C₁₂HClF₂₄O₄S | 2 |
| 5 | H, | 510.9402 | 20.84 | Cl— | C₁₀H₂ClF₁₇O₂ | 4 |
| | Cl—PFCAs | 560.9359 | 22.56 | Cl—/C₂HF₄— | C₁₁H₂ClF₁₉O₂ | 4 |
| | | 610.9348 | 24.11 | Cl—/C₂HF₄— | C₁₂H₂ClF₂₁O₂ | 4 |
| | | 660.9299 | 25.41 | Cl—/CF₃O—/C₂HF₄—/C₄F₉— | C₁₃H₂ClF₂₃O₂ | 4 |
| | | 760.9219 | 27.60 | Cl—/CF₃O— | C₁₅H₂ClF₂₇O₂ | 4 |
| 6 | PFOTs | 516.9535 | 24.17 | C_{F3}O— | C₁₂H3F₁₇OS | 4 |
| | | 566.9520 | 25.43 | CF3O— | C₁₃H3F₁₉OS | 4 |
| | | 716.9379 | 27.66 | CF₃O—/C₄F₉— | C₁₆H3F₂₅OS | 4 |
| 7 | PFOHs | 534.9665 | 20.28 | C₃F₇— | C₁₀HF₂₁O | 4 |
| | | 584.9607 | 25.46 | CF₃O— | C₁₁HF₂₃O | 4 |
| | | 634.9600 | 26.31 | CF₃O—/C₂F₅— | C₁₂HF₂₅O | 4 |
| 8 | PFEAs | 500.9573 | 23.78 | No matched | C₉HF₁₉O₂ | 4 |
| | | 550.9550 | 24.51 | CF₃O—/C₂F₅O— | C₁₀HF₂₁O₂ | 4 |
| | | 600.9542 | 25.78 | CF₃O— | C₁₁HF₂₃O₂ | 4 |
| | | 650.9473 | 26.58 | C_{3F}7—/C₆F₁₁— | C₁₂HF₂₅O₂ | 4 |
| | | 700.9471 | 27.06 | C₃F₇O—/C₄F₉— | C₁₃HF₂₇O₂ | 4 |
| 9 | Po—PFEAs | 382.9565 | 24.32 | CF₃O— | C₆HF₁₃O₄ | 4 |
| | | 532.9481 | 25.73 | C₂F₅O— | C₉HF₁₉O₄ | 4 |
| | | 582.9460 | 26.41 | C₃F₅O₂—/C₃F₇O—/C₇F₁₅O3— | C₁₀HF₂₁O₄ | 4 |
| | | 632.9376 | 26.62 | C₆F₁₁—/C₇F₁₃O— | C₁₁HF₂₃O₄ | 4 |
| 10 | Po—PFECAs | 442.9468 | 21.79 | CF₃O— | C₇HF₁₃O₇ | 2 |
| | | 508.9345 | 23.30 | CF₃O— | C₈HF₁₅O₈ | 2 |
| | | 574.9245 | 25.33 | No matched | C₉HF₁₇O₉ | 3 |
| | | 640.9183 | 26.57 | No matched | C₁₀HF1₉O₁₀ | 3 |
| | | 706.9099 | 27.61 | CF₃O— | C₁₁HF₂₁O₁₁ | 3 |
| 11 | H—Po—PFECAs | 540.9386 | 21.79 | CF₃O— | C₉H2F₁₆O₈ | 4 |
| | | 590.9353 | 23.77 | CF₃O— | C₁₀H2F₁₈O₈ | 4 |
| | | 640.9337 | 24.68 | CF₃O— | C₁₁H2F₂₀O₈ | 4 |
| | | 690.9327 | 25.76 | CF₃O— | C₁₂H2F₂₂O₈ | 4 |
| 12 | | 576.9197 | 23.78 | CF₃O— | C₁₀HF₁₉O₄S | 4 |
| | | 642.9090 | 25.34 | CF₃O— | C₁₁HF₂₁O₅S | 4 |
| | | 708.9007 | 26.57 | CF₃O— | C₁₂HF₂₃O₆S | 4 |
| 13 | | 562.9418 | 23.50 | CF₃O— | C₁₀F₂₀O₄ | 4 |
| | | 628.9341 | 25.33 | CF₃O— | C₁₁F₂₂O₅ | 4 |
| | | 694.9269 | 26.57 | CF₃O— | C₁₂F₂₄O₆ | 4 |
| 14 | | 524.9417 | 21.79 | CF₃O—/C₂HF₄— | C₁₀F₁₈O₄ | 4 |
| | | 590.9353 | 23.77 | CF₃O—/C₉F₁₉— | C₁₁F₂₀O₅ | 4 |
| | | 722.9187 | 26.57 | CF₃O— | C₁₃F₂₄O₇ | 4 |

In summary, this experiment has assessed the application of SWATH-F in gradient-spiked samples and real population serum samples, comparing its performance with nontarget homologue screening results under the IDA acquisition mode. The results indicate that SWATH-F significantly enhances identification capabilities, with an increase of 276% in the detection of PFASs (from 17 types of PFASs identified by IDA to 64 types by SWATH-F). Furthermore, the screening and identification processes of SWATH-F have been automated to facilitate ease of use for researchers, demonstrating that the SWATH-F technology can serve as an effective tool for screening PFASs in environmental and population samples.

TABLE 5
38 PFASs standards and 9 PFASs internal standards (20 ppb).

| Compound name | Abbreviation | Supplier | Purity |
|---|---|---|---|
| 1,1,2,2,3,3,4,4,5,5,6,6,7,7,8,8, | FOSA | Wellington | >98% |
| 8-Heptadecafluoro-octane-1- | | Laboratories | |
| sulfonamide | | | |
| Sodium 1H,1H,2H,2H- | 6:2 FTSA | Wellington | >98% |
| Perfluorooctane sulfonate | | Laboratories | |
| Sodium 1H,1H,2H,2H- | 10:2 FTSA | Wellington | >98% |
| perfluorododecane sulfonate | | Laboratories | |
| 2H-Perfluoro-2-decenoic acid | FOUEA | Wellington | >98% |
| | | Laboratories | |
| 3-Perfluoroheptylpropanoic | FHpPA | Wellington | >98% |
| acid | | Laboratories | |
| 6:2 PFESA/9Cl-PF3ONS | F-53B | Wellington | >98% |
| | | Laboratories | |
| 8:2 PFESA/11Cl-PF3OUdS | F-53B | Wellington | >98% |
| | | Laboratories | |
| Sodium 8-chloroperfluoro-1- | 8Cl—PFOS | Wellington | >98% |
| octanesulfonate | | Laboratories | |
| 9-Chlorohexadecafluoro- | 9Cl—PFNA | TCI | >98% |
| nonanoic acid | | | |
| 7H-Dodecafluoroheptanoic | | CHIRON | >99% |
| 9H-Perfluorononanoate | 9H-PFNA | CHIRON | >99% |
| 11H-Perfluoroundecanoate | 11H-PFUnDA | CHIRON | >99% |
| Hexafluoropropylene oxide | GenX | Wellington | >98% |
| dimer acid | | Laboratories | |
| N-methylperfluoro-1- | N-MeFOSA-M | Wellington | >98% |
| octanesulfonamide | | Laboratories | |
| N-methylperfluorooctane | N-MeFOSAA | Wellington | >98% |
| sulfonamidoacetic acid | | Laboratories | |
| Potassium perfluoro-4- | PFECHS | Wellington | >98% |
| ethylcyclohexanesulfonate | | Laboratories | |
| (isomeric mixture) | | | |
| Sodium | 8:8 PFPi | Wellington | >98% |
| bis(perfluorooctyl)phosphinate | | Laboratories | |
| Sodium bis[2-(N- | diSAmPAP | Wellington | >98% |
| ethyperfluorooctane- | | Laboratories | |
| 1-sulfonamido)ethyl] | | | |
| phosphate | | | |
| Sodium dodecafluoro-3H-4,8- | NaDONA | Wellington | >98% |
| dioxanonanoate | | Laboratories | |
| Perfluorohexanoate | PFHxA | Wellington | >98% |
| | | Laboratories | |
| Perfluoroheptanoate | PFHpA | Wellington | >98% |
| | | Laboratories | |
| Perfluorooctanoate | PFOA | Wellington | >98% |
| | | Laboratories | |
| Perfluorononanoate | PFNA | Wellington | >98% |
| | | Laboratories | |
| Perfluorodecanoate | PFDA | Wellington | >98% |
| | | Laboratories | |
| Perfluoroundecanoate | PFUnDA | Wellington | >98% |
| | | Laboratories | |
| Perfluorododecanoate | PFDoDA | Wellington | >98% |
| | | Laboratories | |
| Perfluorotridecanoate | PFTrDA | Wellington | >98% |
| | | Laboratories | |
| Perfluorotetradecanoate | PFTeDA | Wellington | >98% |
| | | Laboratories | |
| Perfluorohexadecanoate | PFHxDA | Wellington | >98% |
| | | Laboratories | |
| Perfluorooctadecanoate | PFODA | Wellington | >98% |
| | | Laboratories | |
| Perfluorobutane sulfonate | PFBS | Wellington | >98% |
| | | Laboratories | |
| perfluoropentanesulfonate | PFPeS | Wellington | >98% |
| | | Laboratories | |
| Perfluorohexane sulfonate | PFHxS | Wellington | >98% |
| | | Laboratories | |
| Perfluoroheptane sulfonate | PFHpS | Wellington | >98% |
| | | Laboratories | |
| Perfluorooctane sulfonate | PFOS | Wellington | >98% |
| | | Laboratories | |
| Perfluorononane sulfonate | PFNS | Wellington | >98% |
| | | Laboratories | |
| Perfluorodecane sulfonate | PFDS | Wellington | >98% |
| | | Laboratories | |
| Perfluorododecane sulfonate | PFDoDS | Wellington | >98% |
| | | Laboratories | |

In addition to providing a method for nontarget identification of homologues, the disclosure also provides a system for nontarget identification of homologues, including:
an extraction module: configured for performing deconvolution on sample data from mass spectrometry by the sample correlation-based deconvolution algorithm and extracting the sample peaks according to the homologue rule;
an identification module: configured for identifying the perfluoroalkyl substances PFASs by MetFrag after ion annotation is performed on the sample peaks.

The system is mainly composed of the above-mentioned modules. In specific implementation, the above-mentioned modules can be realized as independent entities, or they can be combined arbitrarily to be realized as the same or several entities. For the specific implementation of the above-mentioned units, please refer to the previous method embodiments, and will not be repeated here.

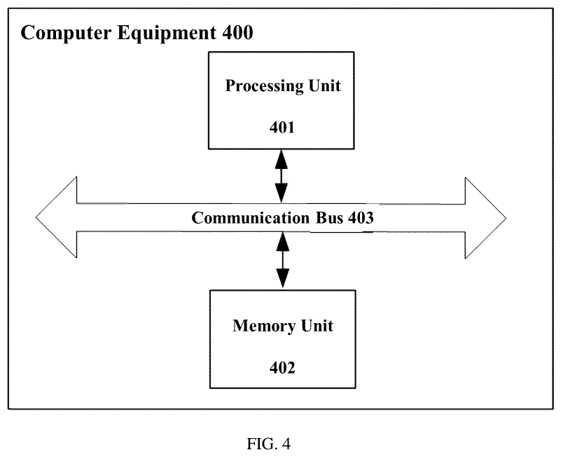

FIG. 4 is a schematic structural diagram of a computer device according to this disclosure. As shown in FIG. 4, the computer device 400 includes at least a memory 402 and a processor 401. The memory 402, connected to the processor 401 via a communication bus 403, stores computer instructions executable by the processor 401. The processor 401 is used to read the instructions of the computer from the memory 402 to realize the steps of the nontarget homologue identification method described in any of the above embodiments.

For the device embodiments described, since they fundamentally correspond to the method embodiments, reference can be made to the descriptions in the method examples for related details. The device embodiments outlined are merely illustrative. The units described as separate components may or may not be physically separate, and the components shown as units may or may not be physical units, meaning they could be located in one place or distributed across multiple network units. Parts or all of the modules might be selected based on actual needs to achieve the objectives of this disclosure. It is understandable and implementable by those skilled in the art without creative efforts.

Suitable computer-readable media for storing computer program instructions and data include all forms of non-volatile memory, media, and memory devices, such as semiconductor memory devices (e.g., EPROM, EEPROM, flash devices), disks (e.g., internal hard disks or removable disks), magneto-optical disks, and CD-ROM and DVD-ROM disks. The processor and memory may be supplemented or integrated into dedicated logic circuits.

Finally, it should be noted that: although this specification contains many specific implementation details, these should not be construed as limiting the scope of any disclosure or the range of protection sought. Instead, these details describe certain features of specific embodiments of the disclosure. Certain features described in multiple embodiments can also be implemented in combination in a single embodiment.

Conversely, various features described in a single embodiment can be implemented separately or in any suitable subcombination in multiple embodiments. Moreover, although features may act in certain combinations as described and even initially claimed as such, one or more features from a claimed combination can in some cases be excised from the combination, and the claimed combination may direct to a subcombination or a variant of a subcombination.

Similarly, while operations are depicted in a specific order in the drawings, this should not be understood as requiring that such operations be performed in the specified order or sequentially, or that all illustrated operations be performed, to achieve desirable results. In certain circumstances, multitasking and parallel processing may be advantageous. Furthermore, the separation of various system modules and components in the described embodiments should not be interpreted as necessary in all embodiments, and it should be understood that the described program components and systems can generally be integrated into a single software product or packaged into multiple software products.

Thus, specific embodiments of the subject have been described. Other embodiments within the scope of the attached claims are possible. In some instances, actions recorded in the claims may be executed in a different order and still achieve desirable results. Additionally, the processes depicted in the figures are not necessarily required to be performed in the exact order shown or sequentially to achieve the desired results. In some implementations, multitasking and parallel processing may prove beneficial.

The descriptions provided herein are intended only preferred embodiments of the disclosure and should not be used to limit the scope of the disclosure. Any modifications, equivalent substitutions, improvements, etc., made within the spirit and principles of the disclosure are intended to be included within the scope of protection of the disclosure.

What is claimed is:

1. A method for nontarget identification of homologues, comprising:
performing deconvolution on sample data from mass spectrometry by a sample correlation-based deconvolution algorithm, extracting sample peaks according to homologue rule, identifying perfluoroalkyl substances PFASs by MetFrag after ion annotation being performed on the sample peaks;
wherein the extracting sample peaks according to homologue rules comprises:
if a mass difference between two peaks is equal to (CF2)n, (CF2O)n, or (CH2CF2)n, the two peaks are retained in a homologue candidate list;
a increasing trend of m/z and retention time is calculated in each homologue series, with an increase of the m/z, the retention time increases, according to mass defect rules of PFAS structural units, characteristic peaks with two decimal places greater than 0.85 or less than 0.15 are retained in a potential PFAS homologue list;
wherein a method for the ion annotation comprises:
building a database comprising 3,496 PFASs and 389 MS2 spectrograms, implementing the ion annotation by Python based on information of the database to obtain 91 characteristic ions, 7 auxiliary identification ions, and 9 types of neutral losses, predicting unknown structures by CFM-ID and SIRIUS;
wherein performing judgment by combining annotated MS2 and the retention time during identifying the perfluoroalkyl substances.

2. An identification system for the method of nontarget identification of homologues according to claim 1, wherein comprises:
an extraction module: configured for performing deconvolution on sample data from mass spectrometry by the sample correlation-based deconvolution algorithm and extracting the sample peaks according to the homologue rule;
an identification module: configured for identifying the perfluoroalkyl substances PFASs by MetFrag after ion annotation is performed on the sample peaks;
wherein the extracting sample peaks according to homologue rules comprises:
if the mass difference between two peaks is equal to (CF2)n, (CF2O)n, or (CH2CF2)n, the two peaks are retained in the homologue candidate list;
the increasing trend of m/z and retention time is calculated in each homologue series, with the increase of the m/z, the retention time increases, according to mass defect rules of PFAS structural units, characteristic peaks with two decimal places greater than 0.85 or less than 0.15 are retained in the potential PFAS homologue list;
wherein a method for the ion annotation comprises:
building the database comprising 3,496 PFASs and 389 MS2 spectrograms, implementing the ion annotation by Python based on information of the database to obtain 91 characteristic ions, 7 auxiliary identification ions, and 9 types of neutral losses, predicting unknown structures by CFM-ID and SIRIUS;
wherein performing judgment by combining annotated MS2 and the retention time during identifying the perfluoroalkyl substances.

3. A computer-readable storage medium on which a computer program is stored, wherein when the program is executed, steps of the method for nontarget identification of homologues according to claim 1 are implemented.

4. A computer device, comprising a memory, a processor, and a computer program stored on the memory and executable on the processor, wherein when the processor executes the program, steps of the method for nontarget identification of homologues according to claim 1 are implemented.
